(19) United States
(12) Patent Application Publication
(10) Pub. No.: US 2024/0317791 A1
(43) Pub. Date: Sep. 26, 2024

(54) A PROCESS FOR THE PRODUCTION OF AMCIPATRICIN DIASCORBATE AND THE USE OF THE SAME FOR THE TREATMENT OF INVASIVE FUNGAL INFECTIONS RESISTANT TO TRADITIONAL ANTIFUNGAL AGENTS

(71) Applicant: BIOSEUTICA B.V., ZEEWOLDE BD (NL)

(72) Inventors: Valerio Maria Ferrari, Milano (MI) (IT); Maria Carla Baggio, Saronno (VA) (IT); Mario Vigilante, Como (IT); Alessandro Calizzi, Malnate (VA) (IT); Tiberio Bruzzese, Milano (IT); Paride Grisenti, Milano (IT)

(73) Assignee: BIOSEUTICA B.V., Zeewolde, BD (NL)

(21) Appl. No.: 18/577,782

(22) PCT Filed: Jul. 8, 2022
(86) PCT No.: PCT/EP2022/069172

§ 371 Date: Jan. 9, 2024

Related U.S. Application Data
(60) Provisional application No. 63/220,598, filed on Jul. 12, 2021.

(51) Int. Cl. C07H 17/08; A61K 9/00; A61K 31/7048; A61K 45/06; A61K 47/26; A61P 31/10; C07H 1/06

(52) U.S. Cl. CPC C07H 17/08; A61K 9/0075; A61K 31/7048; A61K 45/06; A61K 47/26; A61P 31/10; C07H 1/06

(57) ABSTRACT

The invention relates to a process for the production of amcipatricin diascorbate and the use of the same for an improved treatment of invasive fungal infections having proof/prevalence of resistance to traditional antifungal agents.

### SUMMARY OF THE INVENTION

The present invention relates to a process for the production of amcipatricin diascorbate and the use of the same for an improved treatment of invasive fungal infections having proof/prevalence of resistance to traditional antifungal agents.

### BACKGROUND OF THE INVENTION

Invasive fungal diseases are a public health threat and remain currently an important cause of morbidity and mortality. Fungal diseases kill more than 1.7 million and affect over a billion people. Serious fungal infections occur as a consequence of different health problems including asthma, AIDS, cancer, organ transplantation and corticosteroid therapies. In the past many invasive fungal infections were associated with a poor prognosis, because effective therapeutic options were limited. The discovery of new therapeutically active antifungal agents with novel mechanisms of action expanded the number of drugs available over traditional treatment options and have provided clinicians with therapeutic alternatives.

Amphotericin B has been the gold standard for systemic antifungal therapy since its release in the 1950s (Gallis et al. Rev Infect Dis 1990; 12:308-29). This substance belongs to the class of polyene drugs which also include nystatin, which compared to amphotericin B is however more toxic. The polyene agents exert their antifungal activity via binding to ergosterol in the fungal cell membrane. This interaction disrupts cell permeability and results in rapid cell death. Amphotericin B and griseofulvin remained the unique systemic therapeutic options for invasive fungal disease until the early 1970s, when flucytosine, a pyrimidine analogue that inhibit the DNA and protein synthesis in the fungal cell, was approved. The toxicity of griseofulvin and the rapid development of resistance, when used as monotherapy, have limited its routine use for the treatment of invasive fungal infections. In 1978, the first systemic azole broad spectrum antimycotic agent, ketoconazole, was discovered. Azole agents exert their antifungal activity by blocking the demethylation of lanosterol, thereby inhibiting ergosterol synthesis. Ketoconazole was followed chronologically by itraconazole (1980), fluconazole (1982), voriconazole (1991) and posaconazole (1995). The expanded-spectrum of triazoles are endowed of fungicidal activity against a wide spectrum of moulds, as well against Candida species and other yeasts. The echinocandins represent a new class of antifungals. Caspofungin belongs to this class, discovered in 1994, followed by anidulafungin (1993) and micafungin (1996). The mechanism of action of the echinocandins is the inhibition of the production of (1→3)-β-D-glucan, an essential component in the fungal cell wall.

All of the above listed drugs available for treatment of invasive fungal infections have advantages and disadvantages. Amphotericin B is still the most widely used drug for the treatment of fungal infections and for serious disseminated dimorphic fungal and yeast infections caused by Blastomyces, Candida, Cryptococcus and Histoplasma sp. Amphotericin B has been reported to cause nephrotoxicity, reduction of renal blood flow, nausea, vomiting and anorexia, moreover the water solubility of amphotericin B is extremely poor (<0.001 mg/ml in distilled water). Nystatin is too toxic to be used systemically but is used in cases of mucous membrane candidiasis. Griseofulvin is used for the treatment of certain dermatophyte infections caused by Epidermophyton, Microsporum and Trichophyton sp but can cause hepatotoxicity and gastrointestinal distress. The major disadvantage of azoles is the frequent incidence of toxicity, including hepatic necrosis and abdominal cramping. As a consequence, azoles are mainly used topically in the treatment of candidiasis, coccidian meningitis, cutaneous dermatophytes and histoplasmosis. In addition, these drugs are known to interact with a large number of drugs and result in hepatotoxicity. Another potential limitation of azoles is the emergence of Candida sp resistant to fluconazole. Caspofungin has demonstrated activity against Aspergillus sp and Candida sp and is well tolerated, however, it has common side effects, including fever, phlebitis, headache and rash. Limitations of these available antifungal antibiotics all drive the search for novel and safe broad-spectrum antifungal antibiotics.

The therapeutic use of these agents confirmed the need for increased awareness of the antibiotic resistance associated with their use. So, for example, types of fungi, like Candida auris, can become resistant to the above-mentioned class of polyene agents, to echinocandins and to pyrimidine and azole derivatives making fungal infections difficult to treat (Lee, Wee Gyo et al., Journal of Clinical Microbiology (2011), 49(9), 3139-3142).

First detected in 2009, Candida auris is a species of ascomycetous fungus of the genus Candida that grows as a yeast. Most C. auris infections are treatable with echinocandins. However, some C. auris infections are resistant to the main classes of antifungal medications (Chowdhary, Anuradha; et al, Emerging Infectious Diseases (2013), 19(10), 1670-1673. Kean, Ryan et al., mSphere (2019), 4(4), e00458-19/1-e00458-19/10). In this situation, multiple classes of antifungals at high doses may be required to treat the infection but even after treatment for invasive infections, patients usually remain colonized with C. auris for long periods. Candida auris is nowadays a fungus that presents a serious global health threat not only because it is resistant to multiple antifungal drugs commonly used to treat Candida infections but also as it is difficult to identify with standard laboratory methods (misidentification may lead to inappropriate management) and it has caused outbreaks in healthcare settings (Ben-Ami, Ronen et al Emerging Infectious Diseases (2017), 23(2), 195-203).

Among fungal infections mucormycosis, also known as zygomycosis or black fungus, is another serious but rare fungal infection, which generally occurs in immunosuppressed subjects, poorly controlled diabetes and high dose corticosteroid treatments. This fungal infection is caused by fungi in the Mucorales order. In some cases, this infection may be fatal and it is due to an invasion of fungus of the genus Rhizopus (the most common), Mucor, Lichterman, Apophysomyces, Cunninghamella, Mortierella, and Saksenaea. The spores of these fungus are commonly present in the environment (for example on mouldy bread and fruit) and are breathed in frequently but cause disease only in some people.

The pharmacological treatment of mucormycosis involves the use of amphotericin B, initially given slowly into a vein, then given daily for two weeks or alternatively with azoles (isavuconazole or posaconazole). Surgical removal of the “fungus ball” is then indicated.

In early 2021 about 12,000 cases of “black fungus” have been reported in India, mostly in patients recovering from Covid-19, most probably due to over-prescription of steroids used to treat Covid-19 patients. In fact, steroids are prescribed to help ward off the excessive inflammatory response (the so called “cytokine storm”) that hurts the body without stopping the infection caused by the coronavirus. India and Pakistan had the highest rates with around 140 cases per million annually.

Mucormycosis is frequently a life-threatening infection. A review of published mucormycosis cases found an overall all-cause mortality rate of 54% (Roden MM et al., Clin Infect Dis. 2005 Sep. 1;41(5):634-53). The need for more active antimycotic agents for the treatment of “black fungus” stimulated the pharmacological research in this field, since experts consider that the administration of too many steroids to treat Covid-19 could trigger secondary infections and antibiotic resistance to traditionally used antimycotic agents.

Amcipatricin diascorbate (chemical name: Candicidin D, 18-decarboxy-40-demethyl-3,7-dideoxo-N3′-[(dimethylamino)acetyl]-18-[[[2-(dimethylamino)ethyl]-amino]carbonyl]-3,7-dihydroxy-N47-methyl-5-oxo-, cyclic 15,19-hemiacetal, compound with L-ascorbic acid (1:2); registry number 202748-83-2; also coded as SPK-843, SPA-843 or SPA-S-843; other names SPK-843; SPA-843, SPA-S-843; FIG. 1) is known since 1992 (EP 489308).

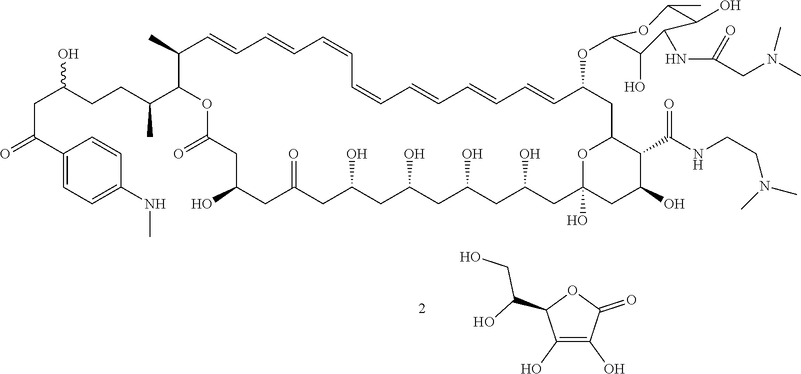 [SMILES: CNc1ccc(C(=O)CC(O)CC[C@H](C)C2OC(=O)C[C@H](O)CC(=O)C[C@H](O)C[C@H](O)C[C@H](O)C[C@H](O)C[C@]3(O)C[C@H](O)[C@@H](C(=O)NCCN(C)C)C(C[C@@H](OC4OC(C)C(O)C(NC(=O)CN(C)C)C4O)/C=C/C=C/C=C/C=C\C=C/C=C/C=C/[C@@H]2C)O3)cc1 ; O=C1O[C@H](C(O)CO)C(O)=C1O]

### Structural Formula of Amcipatricin Diascorbate

This compound is a synthetic amide derivative of partricin A (chemical name: Candicidin D, 40-demethyl-3,7-dideoxo-3,7-dihydroxy-N47-methyl-5-oxo-, cyclic 15,19-hemiacetal; compound identified by the registry number 76551-64-9; FIG. 2) and has fungicidal minimum inhibitory concentration against Candida albicans of 7.5 ng/ml and haemolytic minimum concentration against rat erythrocytes of 18 mg/mL (Bruzzese, T. et al European Journal of Medicinal Chemistry (1996), 31(12), 965-972).

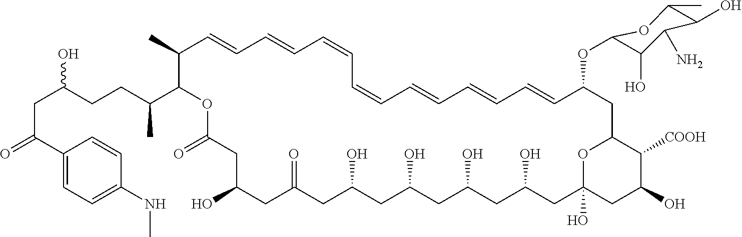 [SMILES: CNc1ccc(C(=O)CC(O)CC[C@H](C)C2OC(=O)C[C@H](O)CC(=O)C[C@H](O)C[C@H](O)C[C@H](O)C[C@H](O)C[C@]3(O)C[C@H](O)[C@@H](C(=O)O)C(C[C@@H](OC4OC(C)C(O)C(N)C4O)/C=C/C=C/C=C/C=C\C=C/C=C/C=C/[C@@H]2C)O3)cc1]

### Structural Formula of Partricin A

The antimicrobial spectrum of amcipatricin diascorbate has been further confirmed against several yeasts and also on different species of Candida including Candida glabrata, Candida krusei, and Candida tropicalis (Rimaroli et al; Antimicrobial Agents and Chemotherapy, 42, 11, 3012-3013, 1998; U.S. Pat. No. 5,908,834). The toxicity of amcipatricin diascorbate was evaluated on different mammalian cell lines and on myeloid committed progenitors. The results show that amcipatricin diascorbate can be considered as prophylactic agent in preventing yeast contamination in cell cultures and also be able to cure cell cultures infected by fungi. The activity of amcipatricin diascorbate compared to that of amphotericin B, a chemically related compound (FIG. 3), against 13 strains of Aspergillus spp., 4 strains of Mucor sp., 4 strains of Rhizopus oryzae, 2 strains Paecilomyces variotii, 5 strains of Penicillium spp., 1 strain of Sporothrix schenkii, 7 strains of Trichophyton spp. And 2 strains of Microsporum spp showed that amcipatricin diascorbate was most fungicidal against Mucor sp. And P. variotii. Amcipatricin diascorbate and amphotericin B showed the same fungicidal activity against Aspergillus spp. (geometric means of the minimum fungicidal concentrations of 12.53 μg/mL), Penicillium spp. (about 12 μg/mL) and S. schenkii (MFC 19.2 μg/mL). Amphotericin B presents geometric means of the minimum fungicidal concentration values lower than those of amcipatricin diascorbate against R. oryzae, Microsporum spp. and Trichophyton spp.

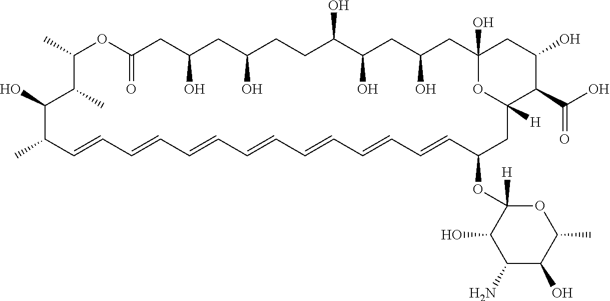 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)O]

### Structural Formula of Amphotericin B

The ability of amcipatricin diascorbate to inhibit Candida sp. conversion from yeast to mycelial form is evident at drug concentrations of 0.25-0.62 mg/L (Strippoli, et al. Journal of Antimicrobial Chemotherapy, 45, 2, 235-237, 2000). The in vitro fungicidal and fungistatic activities of amcipatricin diascorbate were compared with those of amphotericin B against clinically significant Candida sp (n=109), Cryptococcus neoformans (n=49) and Aspergillus spp (n=36) isolates (Kantarcioglu, et al. Journal of Chemotherapy Volume: 15, 3, 296-298, 2003). Single-and multiple-administration trials in rats were performed (Bruzzese et al., Chemotherapy, 47, 2, 77-85, 2001) to assess the serum and tissue concentrations of amcipatricin diascorbate. A dose of 1.25 mg/kg by intravenous route was used both for the single- and multiple-administration trials. The single-administration trial was carried out in comparison with amphotericin B at intravenous doses of 1 mg/kg. Plasma samples were drawn at intervals from 15 min to 96 h after injection.

The elimination half-lives were 22.15 and 18.15 hours, and the area under the curve to infinity (AUC(0-∞)) values were 35.52 and 10.33 μg.hour.ml⁻¹, respectively, for amcipatricin diascorbate and amphotericin B. Both drugs showed an extensive tissue distribution, with higher uptake by the kidneys, followed by the liver, spleen and lungs for amcipatricin diascorbate, and higher uptake by the spleen, followed by the lungs, liver and kidneys for amphotericin B. The multiple-administration trial (1.25 mg/kg/day for 7 days) led to sustained serum and tissue concentrations. On the seventh day, the rats were bled at intervals from 5 min to 96 h after dosing. The serum elimination half-life and AUC(0-00) values were roughly twice those of the single-dose study (41.4 h and 72.1 μg.h.ml⁻¹. respectively). Also, the half-life and AUCs from 0 to infinity of tissues were greater than those in the single-dose trial.

A review on amcipatricin diascorbate for the potential treatment of systemic fungal infections was published in 2005 (Kasanah et al. Current Opinion in Investigational Drugs (Thomson Scientific), 6, 8, 845-853, 2005).

Despite the promising preliminary in vitro and in vivo tests carried out on amcipatricin diascorbate which confirmed a broad-spectrum of antifungal activity at least equal to that of amphotericin B, but with less toxicity, this compound did not enter in therapy for several reasons including chemical, technological reasons and the need to reach a better definition the antimycotic spectrum of amcipatricin diascorbate respect to amphotericin B.

The chemical and technologic difficulties in scaling up the original production process include: low overall yields, low purity, high costs of production, the use of highly toxic, explosive reagents and the fact that the final product can be isolated only through several chromatographic purification and exists only in an amorphous form (not crystalline).

The original production process disclosed by Bruzzese et al. (Eur J Med Chem, 1996, 31, 965-972) carried out the synthesis of SPK-843 base (amcipatricin) using as starting material partricin A in two synthetic steps. This synthetic approach showed some drawbacks for a scale up of the same: low molar yields, low chromatographic purity of the obtained product (the Authors could not obtain a product with more than 95% of purity by HPLC), the use of several chromatographic purifications (three different chromatographic purifications are described) and the use of a toxic and explosive reagent (diphenylphosphoryl azide; compound identified by the registry number 26386-88-9). Particularly amcipatricin base, with the above mentioned HPLC profile, when submitted to a further chromatographic purification in the same experimental conditions disclosed by Bruzzese at al. was recovered with unchanged chromatographic profile; moreover, any attempt to purify amcipatricin base or amcipatricin diascorbate by crystallization failed, giving a product with an unmodified chromatographic profile. In order to solve these issues, it was clear the need to develop an alternative process more robust which could provide product with increased chromatographic purity, suitable to be used as a drug substance and avoiding the use of toxic and dangerous reagents.

The antimycotic effects of amcipatricin diascorbate against known antibiotic resistant Candida strains, like C. auris and against the fungi of the Mucorales genus has been further evaluated in view of its better antimycotic activity against C. albicans, C. krusei, C. flavus and C. niger, better tolerability (lower toxicity) and a good water solubility, when compared to amphotericin B.

In fact, the excellent water solubility of amcipatricin diascorbate (100 mg/ml) allows to define in vivo the optimal pharmacokinetic parameters for an effective treatment of resistant C. auris infections and black fungus infections respect to amphotericin B which, at physiological pH values, is substantially water insoluble and therefore shows a low gastrointestinal absorption rate. Although studies have shown that liposomes as drug carriers can significantly reduce the toxic and side effects of amphotericin B, this alternative formulation is still not fully satisfactory since the antibacterial activity of liposome preparations is lower than that of amphotericin B, therefore the effective therapeutic dose needs to be increased.

Finally, the good water solubility of amcipatricin diascorbate allows to test some unexplored routes of administration of the polyene derivatives in an aqueous medium, like nebuliser solutions, liquid preparations and/or powder for nebuliser solution intended for inhalation use (solution converted into aerosol by a continuously operating nebuliser or a metered-dose nebuliser or pressurised inhalation solution), powder for nebuliser solution as well as in a solid form (Inhalation powder, either in capsules or tablets or other metered dose forms, pre-dispensed, in metered dose forms). For this last approach the possibility to obtain from an aqueous solution, using a spray dry technique, a dry powder with 90% of the particle size distribution equal to or smaller than 5 μm, a particle size capable of penetrating the lung during inhalation, allows to treat fungal infections in the respiratory tract. This particle size cannot be obtained working on the freeze dried powder of amcipatricin diascorbate since this product has low flowability and is highly hygroscopic and therefore not suitable for a micronization procedure.

### SUMMARY OF THE INVENTION

The present invention provides an improved process for the preparation of amcipatricin diascorbate from partricin A. The process of the invention provides in one-pot process the desired compound with 22% overall yields, an HPLC chromatographic purity higher of 97% and avoiding the use of toxic and explosive reagents.

This compound shows a comparable antimycotic activity to fluconazole, voriconazole, posaconazole, amphotericin B and micafungin for the treatment of infections caused by C. auris and by fungal pathogens of the order Mucorales like Mucor circinelloides, Rhizopus oryzae and Rhizopus microspores making therefore amcipatricin diascorbate also potentially active for the treatment of black fungus infections. Moreover, the excellent water solubility of amcipatricin diascorbate allows to obtain, using a spray dry technique, a dry powder with 90% of the particle size equal to or smaller than 5 μm (D90≤5 μm) allowing the development of solid inhaler formulations, which require a reduced particle size in order to obtain an acceptable respirability, as well as liquid formulations to be nebulized, for the treatment of pulmonary fungal infections.

### DETAILED DESCRIPTION

### Preparation of Amcipatricin

The process for the preparation of amcipatricin diascorbate comprises the following steps:
a) preparation of N,N-dimethylglycyl pentafluorophenyl ester from N,N-dimethylglycine, pentafluorophenol and a coupling agent;
b) reaction of N,N-dimethylglycyl pentafluorophenyl ester obtained at step a) with partricin A to afford the intermediate 1;
c) addition of a coupling agent to the reaction mixture containing the intermediate 1 to afford the intermediate 2;
d) addition of N,N′-dimethylaminoethylamine to the reaction mixture containing the intermediate 2;
e) isolation of a precipitate from step d) by aqueous dilution, filtration and drying;
f) purification of the reaction mixture from step e) by direct phase chromatography to afford crude amcipatricin;
g) purification of crude amcipatricin from step f) by reverse phase chromatography to afford purified amcipatricin;
h) addition of L-ascorbic acid to an aqueous solution of amcipatricin from step g) to afford amcipatricin diascorbate;
i) isolation of solid amcipatricin diascorbate by lyophilization or spray drying.

The synthetic scheme of the process of the invention is reported in Scheme 1.

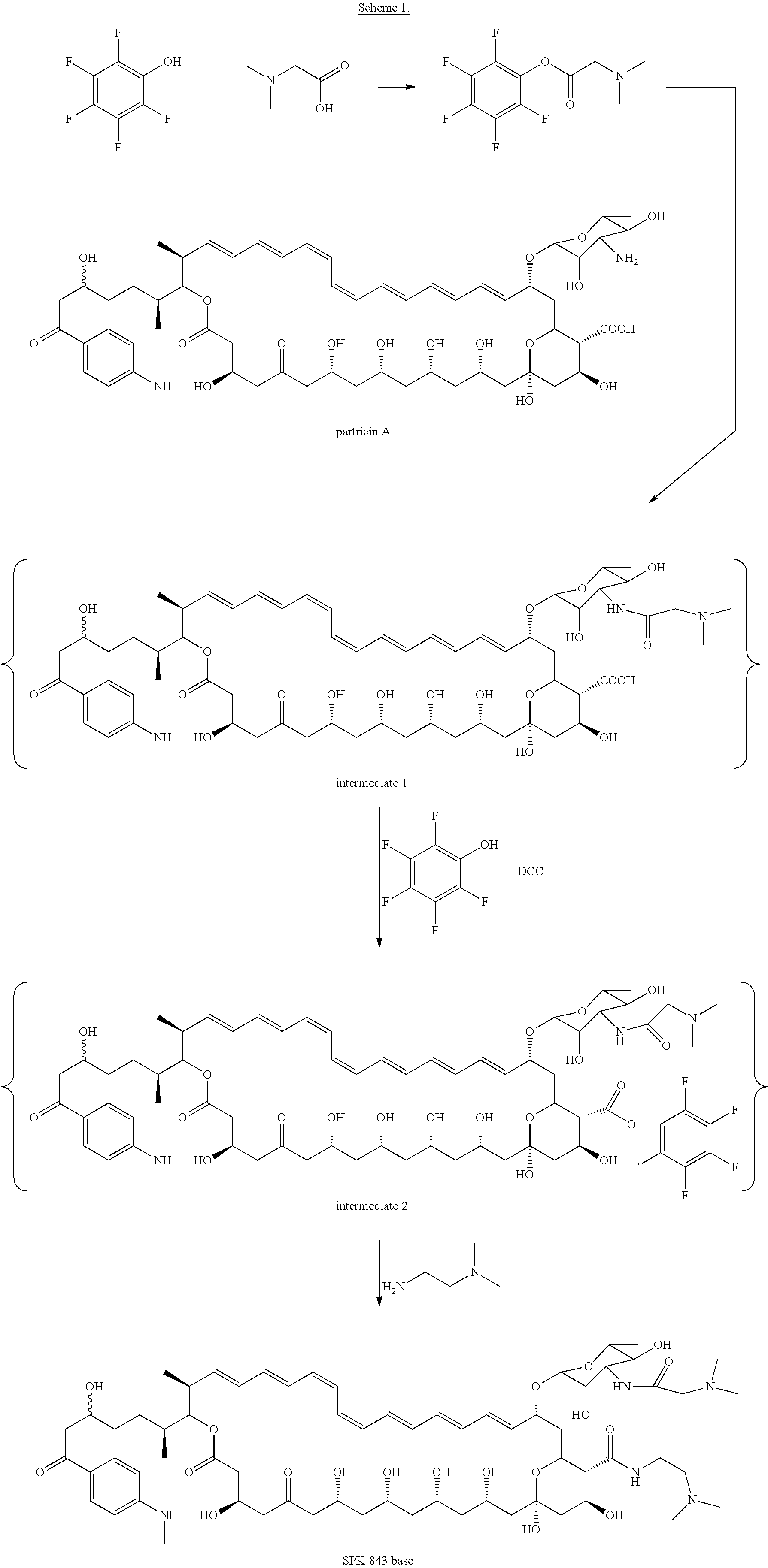

The preparation of N,N-dimethylglycyl pentafluorophenyl ester is effected by reaction of pentafluororophenol in dichloromethane solution with N,N-dimethylglycine hexafluorophosphate azabenzotriazole using a coupling agent, preferably tetramethyluronium (HATU), 1-Ethyl-3-(3-dimethylaminopropyl)carbodiimide hydrochloride (EDC) and N,N′-dicyclohexylcarbodiimide (DCC), more preferably N,N′-dicyclohexylcarbodiimide, in a range of temperature between 15 and 30° C., preferably 20-25° C., for a time ranging between 10 and 34 hours, preferably 24 hours. The pentafluorophenol/N,N-dimethylglycine/N,N′-dicyclohexyl carbodiimide molar ratio is 1.0/1.0=1.2/1.0=1.4, preferably 1.0/1.0/1.3 working concentration of pentafluorophenol comprised in a range of 0.5-2.0 mol/l, preferably 0.78 mol/l.

The preparation of crude amcipatricin was effected by reaction of N,N-dimethylglycyl pentafluorophenyl ester and partricin A in a molecular ratio comprised between 1.0-0.8 and 1.2-1 at a temperature ranging between 15 and 30° C., preferably 25° C., using an aprotic organic solvent with a log P comprised between −0.6 and −1.2, preferably N,N′-dimethylformamide, N,N′-dimethylacetamide or dimethylsulfoxide, at a concentration of partricin A comprised between 0.15 and 0.5 molar, preferably 0.17 molar, for a time ranging between 10 and 60 minutes, preferably 20 minutes. The reaction mixture containing the intermediate 1 (2-(dimethylamino)-N-((2R,3S,4S,5S,6R)-2-(((1R,3S,5S,7R,9R,13R,18S, 19E,21E,23Z,25Z,27E,29E,31E,33R,36R,37S)-36-(hydroperoxy-12-methyl)-1,3,5,7,9,13,37-heptahydroxy-17-((2S,5R)-5-hydroxy-7-(4-(methylamino)phenyl)-7-oxoheptan-2-yl)-18-methyl-11,15-dioxo-16,39-dioxabicyclo[33.3.1]nonatriaconta-19,21,23,25,27,29,31-heptaen-33-yl)oxy)-3,5-dihydroxy-6-methyltetrahydro-2H-pyran-4-yl)acetamide) is then directly treated with a condensing agent selected from hexafluorophosphate azabenzotriazole tetramethyl uronium (HATU), 1-ethyl-3-(3-dimethylaminopropyl)carbodiimide hydrochloride (EDC) and N,N′-dicyclohexylcarbodiimide (DCC), preferably N,N′-dicyclohexylcarbodiimide, to afford the corresponding pentafluoroester intermediate (intermediate 2; perfluorophenyl (1R,3S,5S,7R,9R, 13R, 18S, 19E,21E,23Z,25Z,27E,29E,31E,33R,36R,37S)-33-(((2R,3S,4S,5S,6R)-4-(2-(dimethylamino)acetamido)-3,5-dihydroxy-6-methyltetrahydro-2H-pyran-2-yl)oxy)-1,3,5,7,9,13,37-heptahydroxy-17-((2S,5R)-5-hydroxy-7-(4-(methylamino)phenyl)-7-oxoheptan-2-yl)-18-methyl-11,15-dioxo-16,39-dioxabicyclo[33.3.1]nonatriaconta-19,21,23,25,27,29,31-heptaene-36-carboxylate). The molar ratio of the employed coupling agent to the initial amount of partricin A is comprised in a range of 1/1.5-2.0, preferably 1/1.65. N,N′-Dimethylaminoethylamine is added to this reaction mixture at a temperature ranging between 15 and 40° C., preferably at 20-25° C. The molar ratio of partricin A to N,N′-dimethylaminoethylamine is comprised in a range of 1/4, preferably 1/3.

The reaction mixture is diluted with cool water to directly precipitate crude amcipatricin, which is recovered by suction. The amount of cool water utilized in this step is 3-4 times with respect to the initial amount of aprotic organic solvent used, preferably 3.6 times, and the water temperature is 0-15° C., preferably 10° C.

The resulting crude product (76-80 g) is then purified first by a direct phase silica gel chromatography and then by a reverse phase silica gel chromatography.

The weight ratio of the direct silica gel to the initial amount of partricin A utilized in this purification step is 1:20. A suitable stationary phase utilized in this step is a silica gel with a particle size distribution comprised between 35 and 230 mesh, preferably 70-230 mesh; the mobile phase used in this step consists of three components: an aprotic organic solvent/a polar organic solvent/an aqueous basic solution in 83/15/2 v/v/v volume ratio. Examples of aprotic organic solvents for use as components of the mobile phase include dichloromethane, chloroform, acetone and ethyl acetate, preferably dichloromethane. Examples of polar organic solvents for use as components of the mobile phase include methanol, ethanol, isopropanol, n-propanol, preferably methanol. Suitable aqueous basic solutions which can be used as component of the mobile phase include 10-30% w/v aqueous ammonia solutions, preferably 25% w/v solutions.

The eluted fractions were analysed by HPLC and the selected fractions concentrated under reduced pressure at maximum 35° C. to about the 10-20% of the initial volume to afford a precipitate which is recovered by suction and dried under vacuum at the maximum temperature of 35° C. to afford 13.1 g of crude amcipatricin base (18-decarboxy-40-demethyl-3,7-dideoxo-N3′-[(dimethylamino)acetyl]-18-[[2-(dimethylamino)ethyl]amino]carbonyl]-3,7-dihydroxy-N+7-methyl-5-oxo-, cyclic 15,19-hemiacetal).

Crude amcipatricin is finally purified by reverse phase chromatography. The employed stationary phase can be a reverse phase silica gel RP18 (40-63 μm). The weight ratio of the crude product to the stationary phase is comprised in the range of 1/40-70, preferably 1/65. The crude product is loaded onto the column in an aqueous solution containing L-ascorbic acid: the molar ratio of crude amcipatricin to L-ascorbic acid is comprised in a range of 1/1+7, preferably 1/7, and the concentration of the aqueous solution loaded onto the column, with respect to the moles of crude amcipatricin, is comprised in the range of 1.2-2.0 mol/l. The column is conditioned with an acetonitrile/aqueous L-ascorbate or formate buffer: the volume ratio of acetonitrile to aqueous buffer is comprised in a range of 5-10/95-90, preferably 5/95 and the concentration of the acid is comprised in a range of 10-100 mM, preferably 50 mM. The final pH value of the buffer is comprised in a range from 3.0 to 5.0, preferably 4.1.

The column is eluted at a flow rate of 1 bed volume/5 min increasing gradually the of acetonitrile to buffer ratio from 5-10/95-90 to 30-40/70-60.

The eluted fractions are analysed by HPLC and the selected fractions basified with 5-10% ammonia solution 25% v/v under stirring at 5° C. The final pH value of the selected fractions after addition of the ammonia solution 25% v/v is comprised in a range of 8.0-12.0, preferably 12.0. The obtained solution is maintained under stirring at 0-15° C., preferably 5° C. for 12-18 hours and the precipitated solid is collected by suction and dried under vacuum at a maximum temperature of 35° C. for 16-24 hours to afford 4.3 g of amcipatricin with an HPLC purity >97%.

Amcipatricin diascorbate is prepared from candicidin D, 18-decarboxy-40-demethyl-3,7-dideoxo-N3′-[(dimethylamino)acetyl]-18-[2-(dimethylamino)ethyl]-amino]carbonyl]-3,7-dihydroxy-N47-methyl-5-oxo-, cyclic 15,19-hemiacetal by salt formation with 2-3 equivalent of L-ascorbic acid in an aqueous solution at a concentration of amcipatricin comprised between 0.10-0.20 mol/l, preferably at 0.13 mol/l, at a temperature ranging between 20 and 40° C., preferably 30° C. Solid amcipatricin diascorbate can be isolated from the resulting solution using either the freeze drying or the spray drying technique with ponderal yields from amcipatricin >130%.

Preparation of a Formulation of Amcipatricin Diascorbate with Lactose Monohydrate

The solid formulations of amcipatricin diascorbate with lactose monohydrate of the invention are prepared using the spray dry technique from aqueous solutions of amcipatricin diascorbate in which the concentration of amcipatricin diascorbate in water was comprised in range from 0.5 to 20% w/v, preferably 2%. Lactose monohydrate is added to this solution maintained under stirring, inert atmosphere, cooling a temperature ranging between 0 and 10° C. The weight ratio of amcipatricin diascorbate to lactose monohydrate is comprised in a range from 1/1-3, preferably 1/2.3. The obtained clear product is filtered and the filtrate treated within 1 hour with the spray dryer (temperature inlet 130-150° C.) to afford a formulation of amcipatricin diascorbate with lactose monohydrate with a D90 particle size distribution below 5 μm.

### Experimental Section

### Legenda

MIC: Minimum Inhibitory Concentration (MIC) is defined as the lowest concentration of an antimicrobial ingredient that is bacteriostatic.

D90: in a powder the portion of particles (90%) with diameters below a defined value.

Respirability: the quality or state of being respirable; usually associated for a solid powder inhaler to a particle size capable of penetrating the lung during inhalation (approximately 5 μm and smaller), on a per actuation or per dose basis.

### Preparation of N,N-Dimethylglycyl Pentafluorophenyl Ester

In a round bottom flask methylene chloride (140 mL) and pentafluorophenol (20.1 g) were added. This mixture was maintained under stirring at 20-25° C. to obtain a homogeneous solution, then N,N-dimethylglycine (11.2 g) and N,N′-dicyclohexylcarbodiimide (28.1 g) were sequentially added. The reaction mixture was maintained under stirring at 20-25° C. for 24 hours, then the obtained heterogeneous mixture was filtered under vacuum. The filtrate was evaporated to dryness under reduced pressure to afford 36.8 g of N,N-dimethylglycyl pentafluorophenyl ester, which was used without further purification in the next step.

### Preparation of crude candicidin D, 18-decarboxy-40-demethyl-3,7-dideoxo-N3′-[(dimethylamino)acetyl]-18-|||2-(dimethylamino)ethyl|amino|carbonyl]-3,7-dihydroxy-N47-methyl-5-oxo-, cyclic 15,19-hemiacetal (crude amcipatricin) from partricin A

In a round bottom flask, under stirring at 20-25° C. under a nitrogen flow, N,N′-dimethylformamide (250 mL) and N,N-dimethylglycyl pentafluorophenyl ester (13.8 g) were sequentially added. The obtained mixture was cooled to 10° C. under stirring and partricin A (50 g) was added portion wise. This solution contains intermediate 1 which can be isolated as intermediate or utilized directly in the next step; following this last option the obtained mixture was maintained under stirring at 10° C. for 20′ then N,N′-dicyclohexylcarbodiimide (15 g) was added. The obtained reaction mixture was stirred for 24 hours under nitrogen and the temperature reaction spontaneously increased to 20-25° C. This solution contains intermediate 2 which can be isolated as intermediate or utilized directly in the next step; following this second option N,N′-dimethylaminoethylamine (15 mL) was added dropwise in 60′ to the reaction mixture under stirring at 20-25° C. After 60′ the reaction mixture was added under stirring to water (900 mL) at the temperature of 10° C. and the obtained precipitate recovered by suction and washed on the filter with water (100 mL) and methanol (100 mL). The product was dried in oven under vacuum at +35-40° C. to afford a solid that was suspended under stirring in methanol (0.56 L) at 20-25° C. in a round bottom flask. A 25% ammonia aqueous solution (56 mL) was added dropwise to the obtained suspension. Then methylene chloride (2.24 L) was added and the mixture maintained under stirring for 60′. After this period methylene chloride (2.8 L) and a 25% aqueous ammonia solution (56 mL) were sequentially added and the heterogeneous mixture was filtered by suction. The filtrate was loaded onto silica gel column (70-230 mesh; 1000g) previously conditioned with a dichloromethane/methanol 9/1 v/v mixture. The column was eluted with 8 bed volumes of dichloromethane/methanol/25% aqueous ammonia solution 83/15/2 v/v/v. The eluted fractions were analysed by HPLC and the selected fractions concentrated under reduced pressure at maximum 35° C. to about the 20% of the initial volume.

The resulting heterogenous mixture was then filtered by suction and the obtained wet solid dried under vacuum at maximum 35° C. for 12 hours to afford 13 g of crude amcipatricin.

### Isolation of intermediate 1. 2-(dimethylamino)-N-((2R,3S,4S,5S,6R)-2-(((1R,3S,5S,7R,9R,13R,18S,19E,21E,23Z,25Z,27E,29E,31E,33R,36R,37S)-36-(hydroperoxy-12-methyl)-1,3,5,7,9,13,37-heptahydroxy-17-((2S,5R)-5-hydroxy-7-(4-(methylamino)phenyl)-7-oxoheptan-2-yl)-18-methyl-11,15-dioxo-16,39-dioxabicyclo[33.3.1|nonatriaconta-19,21,23,25,27,29,31-heptaen-33-yl)oxy)-3,5-dihydroxy-6-methyltetrahydro-2H-pyran-4-yl)acetamide

This compound was isolated by chromatographic purification of the reaction mixture containing it. The reaction mixture containing intermediate 1, after evaporation under reduced pressure, was purified by silica gel chromatography (70-230 mesh) using a weight ratio of the obtained residue to silica gel of 1/10. After gradient elution with dichloromethane/methanol pure intermediate 1 was isolated by evaporation under vacuum at 35° C. of the selected fractions.

Elemental analysis calculated for C62H90N3020: theoretical values: C, 62.19; H, 7.58; N, 3.51; 0, 26.72; found values: C, 62.17; H, 7.50; N, 3.49; O, 26.68.

### Isolation of intermediate 2. perfluorophenyl (1R,3S,5S,7R,9R,13R, 18S,19E,21E,23Z,25Z,27E,29E,31E,33R,36R,37S)-33-(((2R,3S,4S,5S,6R)-4-(2-(dimethylamino)acetamido)-3,5-dihydroxy-6-methyltetrahydro-2H-pyran-2-yl)oxy)-1,3,5,7,9,13,37-heptahydroxy-17-((2S,5R)-5-hydroxy-7-(4-(methylamino)phenyl)-7-oxoheptan-2-yl)-18-methyl-11,15-dioxo-16,39-dioxabicyclo[33.3.1]nonatriaconta-19,21,23,25,27,29,31-heptaene-36-carboxylate

This compound was isolated by chromatographic purification of the reaction mixture containing it. The reaction mixture containing intermediate 2, after evaporation under reduced pressure at a temperature below 35° C., was purified by silica gel chromatography (70-230 mesh) using a weight ratio of the obtained residue to the silica gel of 2/10. After gradient elution with dichloromethane/acetone pure intermediate 2 was obtained by evaporation under vacuum at 35° C. of the selected fractions.

Elemental analysis calculated for C69H92F5N3020: theoretical values: C, 60.12; H, 6.73; F, 6.89; N, 3.05; O, 23.21; found values: C, 60.08; H, 6.69; F, 6.82; N, 3.08; O, 23.16.

### Purification of crude candicidin D, 18-decarboxy-40-demethyl-3,7-dideoxo-N^{3′}-[(dimethylamino)acetyl]-18-|||2-(dimethylamino)ethyl|amino|carbonyl]-3,7-dihydroxy-N⁴⁷-methyl-5-oxo-, cyclic 15,19-hemiacetal (crude amcipatricin)

In a round bottom flask L-Ascorbic acid (7.7 g) was dissolved in water (50 mL) then crude amcipatricin (7.7 g) obtained by the previous step was added. The obtained solution was loaded in a column filled with a reversed phase silica gel RP 18 (40-63 μm; 500 g) previously conditioned with an acetonitrile/L-ascorbate buffer (50 mM aqueous solution at pH 4.1) 5/95 v/v mixture. The column was eluted at a flow rate of 100 ml/min using as eluant an acetonitrile/L-ascorbate buffer (50 mM aqueous solution at pH 4.1) 5/95 v/v mixture to an acetonitrile/L-ascorbate buffer (50 mM aqueous solution at pH 4.1) 30/70 v/v mixture. The eluted fractions were analysed by HPLC and the selected fractions basified under stirring at 5° C. with 5-10% ammonia solution 25% v/v. The obtained mixture was maintained under stirring at this temperature for 12-18 hours and the solid precipitated collected by suction and dried under vacuum at maximum 35° C. for 18 hours to afford 4.3 g of amcipatricin base with an HPLC purity >97%.

### Preparation of the diascorbate salt of candicidin D, 18-decarboxy-40-demethyl-3,7-dideoxo-N^{3′}-|(dimethylamino)acetyl]-18-///2-(dimethylamino)ethyl|amino|carbonyl]-3,7-dihydroxy-N⁴⁷-methyl-5-oxo-, cyclic 15,19-hemiacetal (amcipatricin diascorbate)

Into a round bottom flask reactor load demineralized water (480 mL) then nitrogen was bubbled for 30 minutes and, under stirring at the temperature of 30° C., were sequentially added L-ascorbic acid (24.2 g) and amcipatricin (80 g) maintaining the suspension under vigorous stirring to obtain a clear solution. This solution was filtered on a 0.45 μm filter and the filtrate lyophilized to afford 104 g of amcipatricin diascorbate.

Alternatively, amcipatricin diascorbate could be isolated by the above-described solution (about 20% w/v solution), after the filtration on a 0.45 μm filter, as such or after dilution with water till to obtain a 1% w/v concentration, using the spray drying technique at an inlet temperature of 130:150° C.

The chemical-physical properties of the obtained product were in agreement with published literature data.

Preparation of a Formulation of Amcipatricin Diascorbate with Lactose Monohydrate

In a round bottom flask under stirring at 0° C. and under nitrogen atmosphere load amcipatricin diascorbate (2g) and water (200 ml). Maintain this mixture under stirring at 0° C. for 10′ then add portion wise lactose monohydrate (4.6 g) and stir this mixture for additional 10′. The obtained solution is treated within 1 hour with the spray dryer (temperature inlet 130-150° C.) at a feed rate of 3-4 ml/min to afford a formulation of amcipatricin diascorbate with lactose monohydrate 30/70 w/w (5.28 g) with a D90 particle size distribution below 5 μm.

### MIC Determination of Amcipatricin Diascorbate on Different Strains Clinically Isolated of Candida Auris

Testing of the antifungal activity of the compound amcipatricin diascorbate against 22 clinical isolates of the fungal pathogen Candida auris were carried out in order to define the minimal inhibitory concentration (MIC) according to the protocol of the Clinical and Laboratory Standards Institute (CLSI, doc M27,4th edition). Testing has been performed in triplicates for each isolate. The obtained results are reported in Table 1.

TABLE 1
MIC values of amcipatricin diascorbate against 22 clinical isolates of the fungal
pathogen Candida auris and comparison with other antifungal compounds

| | | | MIC values (μg/mL) | | | | |
|---|---|---|---|---|---|---|---|
| | | Clade/Origin/ | | | | | Amcipatricin |
| No | Strains | Reference | Fluconazole | Voriconazole | Amphotericin B | Micafungin | ascorbate |
| 1 | II.2 | II/India | 32 | 1 | 0.25 | 0.125 | 2 |
| 2 | I.2 | I/India | 1024 | 1 | 1 | 0.125 | 2 |
| 3 | I.3 | I/India | 1024 | 2 | 1 | >1024 | 2 |
| 4 | I.4 | I/India | 1024 | 1 | 1 | 0.125 | 2 |
| 5 | II.1 | II/Japan | 4 | 0.0625 | 0.5 | 0.125 | 2 |
| 6 | IV.9 | IV/Israel | 16 | 1 | 0.5 | 0.25 | 2 |
| 7 | IV.10 | IV/Israel | 32 | 0.5 | 0.25 | 0.25 | 2 |
| 8 | IV.11 | IV/Israel | 32 | 0.5 | 0.5 | 0.25 | 1 |
| 9 | IV.12 | IV/Israel | 32 | 1 | 0.5 | 0.25 | 1 |
| 10 | IV.13 | IV/Israel | 32 | 0.5 | 1 | 0.25 | 2 |
| 11 | IV.14 | IV/Israel | 32 | 1 | 0.5 | 0.5 | 2 |
| 12 | III.7 | III/Switzerland | 1024 | 2 | 0.5 | 0.25 | 1 |
| 13 | III.8 | III/Israel | 512 | 2 | 0.5 | 0.25 | 1 |
| 14 | III.9 | III/Israel | 1024 | 2 | 1 | 0.25 | 1 |
| 15 | IV.1 | IV/Colombia | 4 | 0.0625 | 2 | 0.125 | 2 |
| 16 | IV.2 | IV/Colombia | 128 | 1 | 2 | 0.125 | 2 |
| 17 | IV.3 | IV/Colombia | 128 | 1 | 2 | 0.125 | 2 |
| 18 | IV.4 | IV/Colombia | 128 | 0.5 | 1 | 0.125 | 2 |
| 19 | IV.5 | IV/Colombia | 128 | 0.5 | 1 | 0.125 | 2 |
| 20 | IV.6 | IV/Colombia | 128 | 0.5 | 1 | 0.125 | 2 |
| 21 | IV.7 | IV/Colombia | 1024 | 8 | 1 | 0.25 | 2 |
| 22 | IV.8 | IV/Colombia | 1024 | 8 | 1 | 0.25 | 2 |

### MIC Determination of Amcipatricin Diascorbate Against Different ATCC and Clinical Isolates of Fungal Pathogens of the Order Mucorales

Testing of the antifungal activity of amcipatricin diascorbate against different ATCC and clinical isolates of fungal pathogens of the order Mucorales (ATCC and clinical isolated strains), responsible for the fungal infection Mucormycosis, by determination of the minimal inhibitory concentration (MIC), were carried out according to the protocol of the Clinical and Laboratory Standards Institute (CLSI, doc M27,4th edition). Testing has been performed in triplicates for each isolate.

The obtained results collected in the Table 2.

TABLE 2
MIC values of amcipatricin diascorbate against different ATCC
and clinical isolates of fungal pathogens of the order Mucorales
and comparison with other antifungal compounds

| | | | MIC (μg/mL) | | |
|---|---|---|---|---|---|
| | | | Amphotericin | | Amcipatricin |
| Strains | Origin | Sequencing | B | Posaconazole | diascorbate |
| ATCC | ATCC | Mucor | 0.25 | 1.00 | 4 |
| 24905 | strain | circinelloides | | | |
| 2707 | Clinical | Mucor | 0.25 | 0.50 | 4 |
| | strains | circinelloides | | | |
| 2109 | Clinical | Mucor | 0.50 | 0.50 | 4 |
| | strains | circinelloides | | | |
| 4280 | Clinical | Mucor | 1 | 1 | 4 |
| | strains | circinelloides | | | |
| 2086 | Clinical | Rhizopus | 1 | 0.25 | 4 |
| | strains | oryzae | | | |
| 3315 | Clinical | Rhizopus | 0.25 | 0.125 | 2 |
| | strains | microsporus | | | |
| 3435 | Clinical | Rhizopus | 0.50 | 0.50 | 4 |
| | strains | microsporus | | | |

1. A process for preparing amcipatricin diascorbate comprising the following steps:
a) preparing N,N-dimethylglycyl pentafluorophenyl ester from N,N-dimethylglycine, pentafluorophenol and a coupling agent;
b) reacting N,N-dimethylglycyl pentafluorophenyl ester obtained at step a) with partricin A to afford a reaction mixture containing the intermediate 1;
c) adding a coupling agent to the reaction mixture containing the intermediate 1 to afford a reaction mixture containing intermediate 2;
d) adding N,N′-dimethylaminoethylamine to the reaction mixture containing the intermediate 2;
e) isolating a precipitate from step d) by aqueous dilution, filtration and drying;
f) purifying the reaction mixture from step e) by direct phase chromatography to afford crude amcipatricin;
g) purifying crude amcipatricin from step f) by reverse phase chromatography to afford purified amcipatricin;
h) adding L-ascorbic acid to an aqueous solution of amcipatricin from step g) to afford amcipatricin diascorbate;
i) isolating solid amcipatricin diascorbate by lyophilization or spray drying.

2. A process according to claim 1 wherein the coupling agent in steps a) and c) is selected from hexafluorophosphate azabenzotriazole tetramethyl uronium (HATU), 1-Ethyl-3-(3-dimethyllaminopropyl)carbodiimide hydrochloride (EDC) and N,N′-dicyclohexylcarbodiimide (DCC).

3. A process according to claim 1 wherein in step a) the pentafluorophenol/N,N-dimethylglycine/coupling agent molar ratio is 1.0/1.0:1.2/1.0+1.4.

4. A process according to claim 1 wherein N,N-dimethylglycyl pentafluorophenyl ester and partricin A are used in step b) in molar ratio comprised between 1.0/0.8 and 1.2 at a temperature ranging between 15 and 30° C., using an aprotic organic solvent with a log P comprised between −0.6 and −1.2.

5. A process according to claim 4 wherein the aprotic organic solvent is N,N′-dimethylformamide, N, N′-dimethylacetamide or dimethylsulfoxide or a mixture thereof.

6. A process according to claim 1 wherein the molar ratio of partricin A to N,N′-dimethylaminoethylamine in step d) is in a range of 1/3 to 1/4.

7. A process according to claim 1 wherein the stationary phase in step f) is a silica gel with a particle size distribution of between 35 and 230 mesh and the mobile phase is consisting of: an aprotic organic solvent, a polar organic solvent and an aqueous basic solution in 83/15/2 v/v/v volume ratio.

8. A process according to claim 7 wherein the aprotic organic solvent is dichloromethane, chloroform, acetone or ethyl acetate; the polar organic solvent is methanol, ethanol, isopropanol or n-propanol; the aqueous basic solutions comprise 10-30% w/v aqueous ammonia solutions.

9. A process according to claim 1 wherein the stationary phase in step g is a reversed phase silica gel RP18 (40-63 μm), or equivalent; the weight ratio of the crude amcipatricin to the stationary phase is comprised in the range of 1/40-70; the elution is carried out at a flow rate of 1 bed volume/5 min increasing the relative ratio of acetonitrile to the buffer from the range of 5:10-95:90 to 30:40-70:60.

10. A process according to claim 9 wherein the buffer is a L-ascorbate or a formate buffer; the volume ratio of acetonitrile to the aqueous buffer is comprised in a range of 5:10/95:90; the concentration of the acid is comprised in a range of 10-100 mM; the final pH value of the buffer is comprised in a range from 3.0 to 5.0.

11. A process according to claim 1 wherein the salt formation in step h) is effected using 2-3 equivalent of L-ascorbic acid in an aqueous solution working at a concentration of amcipatricin of between 0.10-0.20 mol/l at a temperature ranging between 20 and 40° C.

12. A process according to claim 1 wherein the isolation of amcipatricin diascorbate in step i is effected using the spray drier from 1-20% aqueous solutions working at an inlet temperature of 130-150° C.

13. A process according to claim 12 wherein the obtained particle size distribution DL90 is below 5 μm.

14. The compound 2-(dimethylamino)-N-((2R,3S,4S,5S,6R)-2-(((1R,3S,5S,7R,9R,13R,18S,19E,21E,23Z,25Z,27E,29E,31E,33R,36R,37S)-36-(hydroperoxy-12-methyl)-1,3,5,7,9,13,37-heptahydroxy-17-((2S,5R)-5-hydroxy-7-(4-(methylamino)phenyl)-7-oxoheptan-2-yl)-18-methyl-11,15-dioxo-16,39-dioxabicyclo[33.3.1]nonatriaconta-19,21,23,25,27,29,31-heptaen-33-yl)oxy)-3,5-dihydroxy-6-methyltetrahydro-2H-pyran-4-yl)acetamide of formula 1

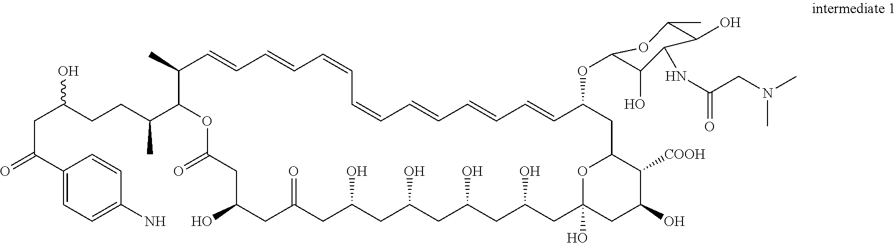 [SMILES: CC1OC(O[C@H]2/C=C/C=C/C=C/C=C\C=C/C=C/C=C/[C@H](C)C([C@@H](C)CCC(O)CC(=O)c3ccc(N)cc3)OC(=O)C[C@H](O)CC(=O)C[C@H](O)C[C@H](O)C[C@H](O)C[C@H](O)C[C@]3(O)C[C@H](O)[C@@H](C(=O)O)C(C2)O3)C(O)C(NC(=O)CN(C)C)C1O]

15. The compound perfluorophenyl (1R,3S,5S,7R,9R,13R, 18S,19E,21E,23Z,25Z,27E,29E,31E,33R,36R,37S)-33-(((2R,3S,4S,5S,6R)-4-(2-(dimethylamino) acetamido)-3,5-dihydroxy-6-methyltetrahydro-2H-pyran-2-yl)oxy)-1,3,5,7,9,13,37-heptahydroxy-17-((2S,5R)-5-hydroxy-7-(4-(methylamino)phenyl)-7-oxoheptan-2-yl)-18-methyl-11,15-dioxo-16,39-dioxabicyclo[33.3.1]nonatriaconta-19,21,23,25,27,29,31-heptaene-36-carboxylate of formula 2

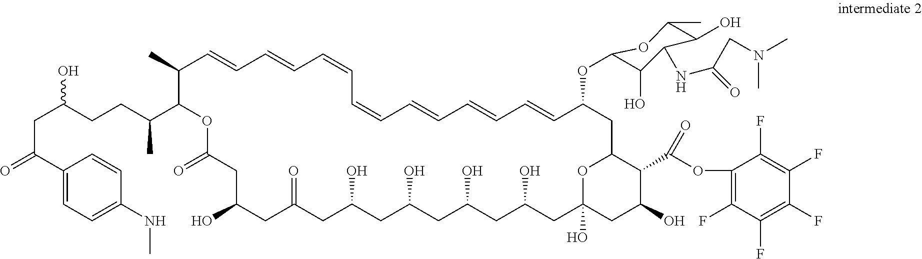 [SMILES: CNc1ccc(C(=O)CC(O)CC[C@H](C)C2OC(=O)C[C@H](O)CC(=O)C[C@H](O)C[C@H](O)C[C@H](O)C[C@H](O)C[C@]3(O)C[C@H](O)[C@@H](C(=O)Oc4c(F)c(F)c(F)c(F)c4F)C(C[C@@H](OC4OC(C)C(O)C(NC(=O)CN(C)C)C4O)/C=C/C=C/C=C/C=C\C=C/C=C/C=C/[C@@H]2C)O3)cc1]

16. A method of treatment of infections caused by C. auris and/or by fungal pathogens of the order Mucorales like Mucor circinelloides, Rhizopus oryzae and/or Rhizopus microspores, with different etiology comprising the administration of an effective amount of amcipatricin diascorbate to subjects in need thereof.

17. A pharmaceutical composition comprising an effective amount of amcipatricin or amcipatricin diascorbate, alone or in combination with other therapeutic agents, formulated with suitable carriers and/or excipients for oral, cutaneous/subcutaneous, buccal, auricular, inhalation, intracardiac, nasal, ocular, vaginal or injectable/infusional administration.

18. A composition according to claim 17 wherein the other therapeutic agents are selected from antibiotic agents, bronchodilator agents, steroids, phosphodiesterase-4 (PDE-4) inhibitors.

19. A composition according to claim 17 as solid formulation of amcipatricin diascorbate with lactose monohydrate with a DL90≤5 μm prepared by spray drying.

20. A composition according to claim 19 in form of dry powder inhaler (DPI) formulations.

21. A composition according to claim 17 in form of liquid metered dose inhaler (MDI).
